(19) United States
(12) Patent Application Publication
(10) Pub. No.: US 2016/0206298 A1
(43) Pub. Date: Jul. 21, 2016

(54) Combination Hemostatic Tablet or Powder and Radial Arterial Compression Band with Syringe Assembly

(71) Applicant: Biolife, L.L.C., Sarasota, FL (US)

(72) Inventors: Talmadge Kelly Keene, Ruskin, FL (US); Timothy J. Kelly, Sarasota, FL (US); John Tifton Fordham, Indialantic, FL (US); Mark Travi, Venice, FL (US)

(73) Assignee: Biolife, L.L.C.

(21) Appl. No.: 15/002,549

(22) Filed: Jan. 21, 2016

Related U.S. Application Data
(60) Provisional application No. 62/251,218, filed on Nov. 5, 2015.
(60) Provisional application No. 62/193,245, filed on Jul. 16, 2015.
(60) Provisional application No. 62/105,845, filed on Jan. 21, 2015.

(51) Int. Cl. A61B 17/00; A61B 17/135; A61B 17/132

(52) U.S. Cl. CPC A61B 17/0057; A61B 17/1325; A61B 17/135; A61B 2017/00659; A61B 2017/00676; A61B 2017/00778; A61B 2017/00884; A61B 2017/12004

(57) ABSTRACT

A combination radial arm band and hemostatic material preferably including potassium ferrate and a cation ion exchange resin in powder or granular form (WOUNDSEAL) or formed into a solid tablet (STATSEAL) and having improved topical effectiveness in the arresting of blood flow from a puncture wound into the radial artery made during transradial diagnostic or interventional catheterization procedures. The combination reduces time and pressure needed to achieve hemostasis and decreases arterial damage as compared to use of a radial arm band alone. In one aspect of this disclosure, careful limitation of applied force and time of applied force against the STATSEAL tablet reduces likelihood of harm to the radial artery while minimizing time to achieve hemostasis.

### CROSS-REFERENCE TO RELATED APPLICATIONS

Not applicable

### STATEMENT REGARDING FEDERALLY SPONSORED RESEARCH OR DEVELOPMENT

Not applicable

### INCORPORATION-BY-REFERENCE OF MATERIAL SUBMITTED ON A COMPACT DISC

Not applicable

### BACKGROUND OF THE INVENTION

### Field of the Invention

This disclosure relates generally to hemostatic products, and particularly to the novel combination of a hemostatic device or material and a radial arterial compression band which, when applied together to a bleeding puncture wound following transradial diagnostic or interventional catheterization procedures, substantially reduce time and/or pressure to achieve hemostasis. An improved syringe for achieving optimal pressure against a bleeding artery puncture to more effectively achieve hemostasis may also be provided.

### DESCRIPTION OF RELATED ART

Hemostasis powders are well known. Thompson et al, U.S. Pat. No. 4,545,974 & 4,551,326, disclose processes for the manufacture of potassium ferrate and similar high oxidation state oxyiron compounds. Patterson et al U.S. Pat. No. 6,187,347 and Patterson et al. U.S. Pat. No. 6,521,265, disclose the mixing of potassium ferrate and anhydrous strongly acidic cation exchange resins for the cessation of bleeding. These patents are incorporated by reference herein in their entirety. This powderous or granular hemostatic mixture has been formed into solid tablet or wafer devices as disclosed in Hen et al., U.S. Pat. Nos. 8,961,479 and 9,039,667, both incorporated herein by reference.

Transradial catheterization is an endovascular procedure or catheterization procedure performed to diagnose and treat arterial disease (e.g., coronary artery disease, peripheral artery disease, etc.). Endovascular procedure can be performed achieving access into body's arterial system from either femoral artery (in groin), brachial artery (in elbow) or radial artery in the wrist. The transfemoral (through groin) approach to perform cardiac catheterization has typically been more prevalent in invasive cardiology. But, radial access has gained in popularity due to technical advances with catheters and lower complication rates than transfemoral access.

In past few years, transradial access for coronary intervention has become increasingly popular. The most advantageous aspect is lower access-site bleeding complications even with aggressive use of anticoagulation and antiplatelet therapies. During the angioplasty and stent procedures, patients are given therapeutic (high) doses of anticoagulation (blood thinners) and platelet inhibiting medications.

With transfemoral access, the rate of bleeding complications is 3%-6%. Occasionally patients can develop retroperitoneal bleeding (bleeding into the pelvic cavity), and up to 1% of patients require blood transfusion to treat the bleeding complication after transfemoral catheterizations. Patients may also develop painful hematoma, A-V fistula or pseudoaneurysms. In modern interventional cardiology, the procedural success rates are high and ischemic complications are relatively rare. However the bleeding complications associated with transfemoral catheterization have not been significantly reduced even after trying new pharmacological strategies. There are strong evidence suggesting that post PCI bleeding is associated with an adverse prognosis. Post-procedural blood transfusion is also associated with poor prognosis. Bleeding complication, pseudoaneurysm, hematoma formation are less than 2% with transradial catheterization. Possibility of blood transfusion requirement is extremely rare after transradial catheterization.

The other reason for the increased use of radial access is the technological advances in the sheath and catheter design and improved physician experience with this approach. With improvement in the physician's experience, radial artery access is now being used with equal efficacy to treat almost every complex coronary artery disease, including acute myocardial infarction, chronic total occlusion, bifurcation coronary artery disease and rotablation. Radial access has also been used successfully to treat peripheral artery disease including bilateral iliac artery stenosis, renal artery stenosis and for carotid interventions.

Due to rapid ambulation post procedure, the radial interventions became particularly attractive for patients with back pain, chronic obstructive lung disease, prostatic hypertrophy and elderly patients. As after catheterization through femoral approach, patient is generally required to lay flat with immobilization of the leg for 4-6 hours. Early ambulation and early discharge after transradial catheterization improves quality of life and reduces morbidity. Both patients and hospital staff typically strongly prefer the transradial approach as opposed to femoral access.

Although transradial procedures often have fewer complications than femoral procedures, they have some shortcomings. The procedure is technically more difficult and has a relatively long learning curve, which includes the potential for unsuccessful completion of procedure during the learning curve. In clinical trials before crossing the learning curve, there is up to 5% failure in completing the catheterization successfully from radial approach. Technical difficulties are because of loops and tortuosity of the radial and the subclavian artery, anatomical variations in radial artery, and radial spasm.

Major complication associated with a transradial interventions include early and late radial artery occlusion. Most of the radial artery occlusions are asymptomatic. Post PCI radial artery occlusion can be reduced by using smaller diameter catheters and anticoagulation. Also by avoiding prolonged compression of the radial artery and applying just enough pressure to achieve hemostasis reduces this complication of asymptomatic radial occlusion significantly. Implying this approaches the radial artery occlusion rate has come down to 1.1-1.8%.

All radial bands on the market today operate by one of the following methods to apply pressure to the radial artery:

1) A balloon held in place with a VELCRO strap and filled with a “volume” of air/water.

2) A strap that is pulled tight with a solid or foam pressure attachment over the artery
a. VELCRO material
b. Zip Tie
c. Elastic band

3) A screw mechanism to apply pressure

The problem with all of these devices is they are relying on a person to apply the band to every patient's arm exactly the same way to achieve a consistent result. The bands are put around the wrist and secured with VELCRO material. Next, the band is inflated to a volume of air to apply pressure to the bleeding site. A medical professional can accurately apply a correct volume of air. However, variation in applying the band will impact the pressure created as the balloon is inflated. These variations are:

1) Size of patient's wrist;

2) How tightly the band is secured to the wrist;

3) Different personnel putting the bands on patients.

Not only will an individual medical professional vary slightly from patient to patient, but different personnel will be applying the bands.

Testing has shown that an 80% difference in force applied to the wrist by the same medical professional, using the same patient's wrist, applying the band twice within 45 minutes. These medical professionals had less than 3 mm difference in the tightness of the band (measured by overhang of the strap). Less than 3 mm of band tightness resulted in nearly a doubling of force applied to close the artery.

The foregoing examples of the related art and limitations related therewith are intended to be illustrative and not exclusive. Other limitations of the related art will become apparent to those skilled in the art upon a reading of the specification and a study of the drawings.

### BRIEF SUMMARY OF THE INVENTION

This disclosure is directed to the combination, in one aspect, of a novel hemostatic device or material marketed by Biolife, L.L.C. under the trademark STATSEAL and a radial arm band. The device as a solid hemostatic tablet is applied to a transdermal puncture wound followed by applying pressure against the puncture wound by applying the radial arm band around the arm and against the hemostatic tablet for a time sufficient to clot and arrest substantial further blood flow from the puncture wound in the radial artery.

In another embodiment, the hemostatic device is in powder or granular form marketed by Biolife, L.L.C. under the trademark WOUNDSEAL with or without magnetite, the pressure pad having a magnet for ease of picking up and holding a quantity of WOUNDSEAL with magnetite.

In another embodiment, the hemostatic device (STATSEAL) is in solid tablet form and the system which applies pressure against the tablet includes regulation of applied pressure so as to carefully limit the force applied against the tablet, puncture, and radial artery to minimize time, pressure, and potential harm to the artery.

All radial arm bands have a pressure pad or surface which applies a variable pressure against the cannula puncture into the radial artery which occurs during transradial diagnostic or interventional catheterization procedures. The combination of the hemostatic device and a radial artery band of this disclosure produces a synergistic shortening of time and applied pressure required to effect hemostasis. Where the pressure interface with the puncture is an inflatable member such as a balloon, a novel syringe arrangement to more accurately regulate applied pressure against the puncture may also be provided.

The following embodiments and aspects thereof are described and illustrated in conjunction with systems, tools and methods which are meant to be exemplary and illustrative and not limiting in scope. In various embodiments one or more of the above-described problems have been reduced or eliminated while other embodiments are directed to other improvements. In addition to the exemplary aspects and embodiments described above, further aspects and embodiments will become apparent by reference to the drawings and by study of the following descriptions.

### BRIEF DESCRIPTION OF THE SEVERAL VIEWS OF THE DRAWING(S)

FIGS. 1A and 1B are perspective and top plan views, respectively, of a round tablet embodiment of the hemostatic device of the combination.

FIGS. 1C and 1D are perspective and top plan views, respectively, of a rectangular embodiment of the hemostatic device.

FIG. 2 is a perspective view of a radial arm band 14 in place on an arm A of a patient showing the position of the radial arm band 14 relative to the radial and ulnar arteries R and U of the arm A.

FIG. 3 is a schematic cross-sectional view of the wrist anatomy of FIG. 2.

FIG. 4 is a side view of an adjustable radial arm band 16 for use in conjunction with the hemostatic tablet 10.

FIG. 5 is a perspective view of another adjustable radial arm band 22 for use in conjunction with the hemostatic tablet 12.

FIG. 6 is a perspective view of another adjustable radial arm band 34 for use in conjunction with the hemostatic tablet 10.

FIG. 7A is a perspective view of another adjustable radial arm compression band 50 for use in combination with the hemostatic device 10.

FIG. 7B is a section view of the plunger 62 of FIG. 7A.

FIG. 8 is a section view showing another radial arm band 70 having an inflatable balloon for applying pressure against the radial artery R with the hemostatic device 10 positioned against the puncture.

FIG. 9 is a perspective view of a femoral compression device 90 for use in combination with the hemostatic tablet 10.

FIG. 10 is a perspective view of another adjustable radial arm band 110 for use in combination with hemostatic tablet 10.

FIGS. 11A and 11B are side elevation and perspective views, respectively, of another adjustable radial arm band 120 in combination with hemostatic tablet 10.

FIGS. 12A to 12C depict use of WOUNDSEAL hemostatic powder with and without magnetite in combination with a radial arm band 140.

FIGS. 13 to 17 show embodiments of the invention which utilize a STATSEAL hemostatic tablet 10 and a radial arm band system 170, 190, 210, 240, or 270 in conjunction with pressure controlling devices which prevent over-pressurization of the hemostatic tablet 10 against the puncture and radial artery R.

FIGS. 18A, 18B, 19A and 19B show other embodiments of syringe assemblies 290 and 320 which utilize a STATSEAL hemostatic tablet 10 in conjunction with a controlled pressure application system incorporating an inflatable balloon for preventing over-pressurization of the STATSEAL tablet against the puncture and radial artery R.

Exemplary embodiments are illustrated in reference figures of the drawings. It is intended that the embodiments and figures disclosed herein are to be considered to be illustrative rather than limiting.

### DETAILED DESCRIPTION OF THE INVENTION

### Nomenclature

10. round hemostatic tablet
12. square hemostatic tablet
13. band member
14. radial arm clamp
15. pad member
16. radial artery compressor
18. adhesive layer
19. support pad
20. compression pad
22. radial artery compression device
24. radial arm band
26. plastic support plate
28. color marker
30. turn cap
32. compression pad
34. radial artery compression device
36. band
38. transparent support plate
40. screw nut
42. air cushion
44. radial artery compression device
50. radial artery compression device
52. footplate
54. footplate
58. rack
60. rack
62. plunger
64. housing
66. footplate
70. radial arm compression device
72. band
74. curved outer plate
76. balloon
78. cavity
80. connector
90. femoral compression device
92. belt
94. base plate
96. belt engaging end
98. belt engaging end
100. support cup
102. Inflatable pressure element
104. pump
110. radial artery compression device
112. strap
114. compression pad
116. adjustment knob
118. band end
120. hemostasis band
122. body component
124. body component
126. hinge
128. pad
130. sensor apparatus
132. balloon
134. fastener component
136. fastener component
140. radial artery hemostatic system
142. support plate
144. plunger tube
146. WOUNDSEAL hemostatic powder
150. WOUNDSEAL hemostatic powder and magnetite
152. plunger
154. pressure pad
156. magnet
158. container
160. radial arm band
170. radial artery hemostatic system
172. radial band assembly
174. radial band
176. support plate
178. plunger housing
180. plunger
182. pressure pad
183. pressure control device
184. relief valve
186. pressure relief port
188. check valve
189. syringe
190. radial artery hemostatic system
192. radial arm band assembly
194. support plate
196. plunger housing
198. plunger
200. calibrated spring
202. pre-activation detent
204. plunger travel limit
206. pressure pad
210. radial artery hemostatic system
224. pressure release
226. syringe
228. pressure regulator
230. tip
232. pressurization port
234. pressurization relief port
240. radial artery hemostatic system
242. radial band assembly
244. radial band
246. support plate
248. balloon
250. pressure regulator assembly
252. syringe
254. adjustable pressure regulator
256. syringe tip
258. pressurization port
260. pressure relief port
262. check valve
264. balloon inlet
266. adjustment knob
270. radial artery hemostatic system
272. pressure regulator assembly
274. syringe
276. adjustable pressure regulator
278. adjustment knob
280. pressure relief port
282. syringe tip
284. pressurization port
286. check valve
290. syringe assembly
292. syringe body
294. syringe pressure tube
296. sight tube
298. movable pressure indicator
300. air transfer passage
302. syringe nozzle
304. air discharge port
306. collar
308. sight chamber
310. syringe chamber
312. plunger
314. plunger seal
316. syringe discharge pressure portion
320. syringe assembly
322. plunger assembly
324. plunger
326. sight pressure tube
328. movable pressure Indicator
330. plunger seal
332. air pressure port
334. sight chamber
336. sight pressure chamber
340. syringe tube
342. syringe chamber
346. syringe nozzle

U.S. application Ser. No. 09/500,902, filed Feb. 9, 2000, now U.S. Pat. No. 6,187,347, issued Feb. 13, 2001, for a hemostatic composition (WOUNDSEAL) for Arresting the Row of Blood and Method is incorporated herein by reference in its entirety.

U.S. application Ser. No. 13/760,319, filed Feb. 6, 2013, now U.S. Pat. No. 8,979,726 for Hemostatic Composition (WOUNDSEAL) with Magnetite is incorporated herein by reference in its entirety.

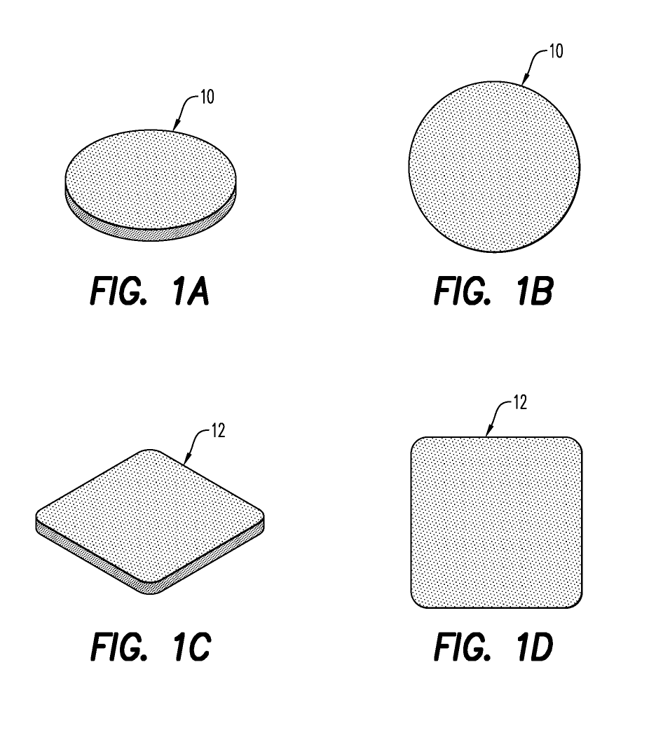

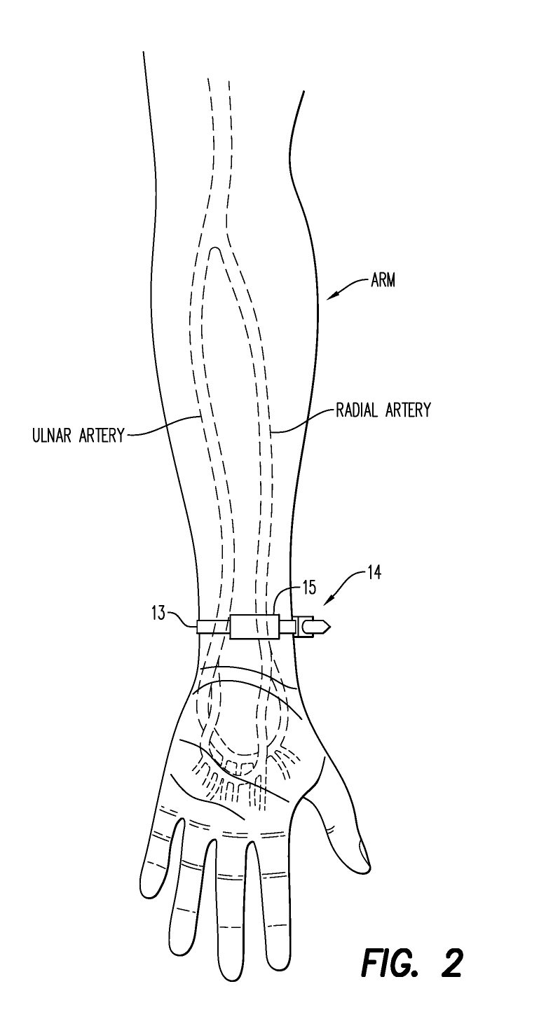

U.S. application Ser. No. 13/847,057, filed Mar. 19, 2013, now U.S. Pat. No. 8,961,479 for Hemostatic Device (STATSEAL) and Method (incorporated herein by reference) are shown in FIGS. 1A through 1D.

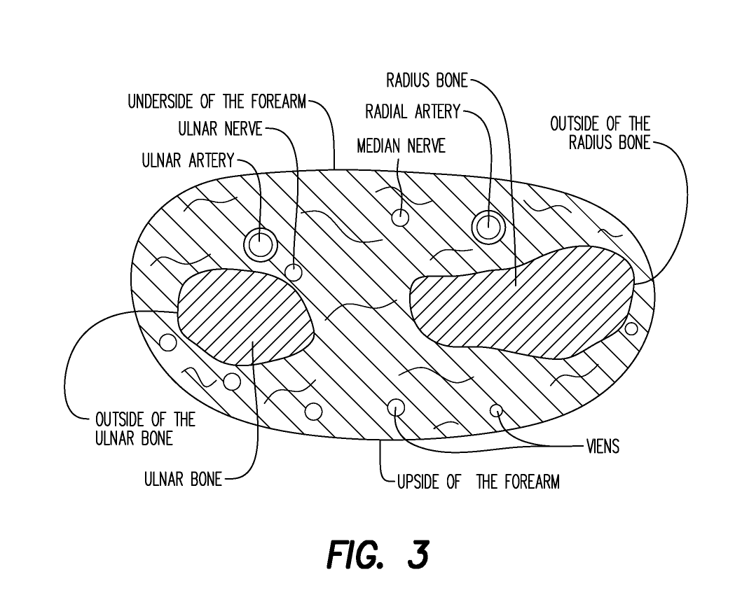

Referring to FIG. 2, a prior art radial arm clamp 14 disclosed in U.S. Pat. No. 4,760,846, includes a band member 13 and a pad member 15. The band member 13 includes a strap approximately 14″ long having an array of parallel transverse ridges formed on its underside surface. A buckle is formed at one end of the strap and includes a slot through which the strap portion can pass. The buckle also includes a tongue having teeth (not shown) which engage the ridges of the strap to permit the clamp to be held in place around the arm A. The tongue is spring biased to bring the teeth into engagement with the ridges. The tongue is moveable, however, for permitting the clinician to disengage the teeth from engagement with the ridges for removal of the clamp from the patients arm.

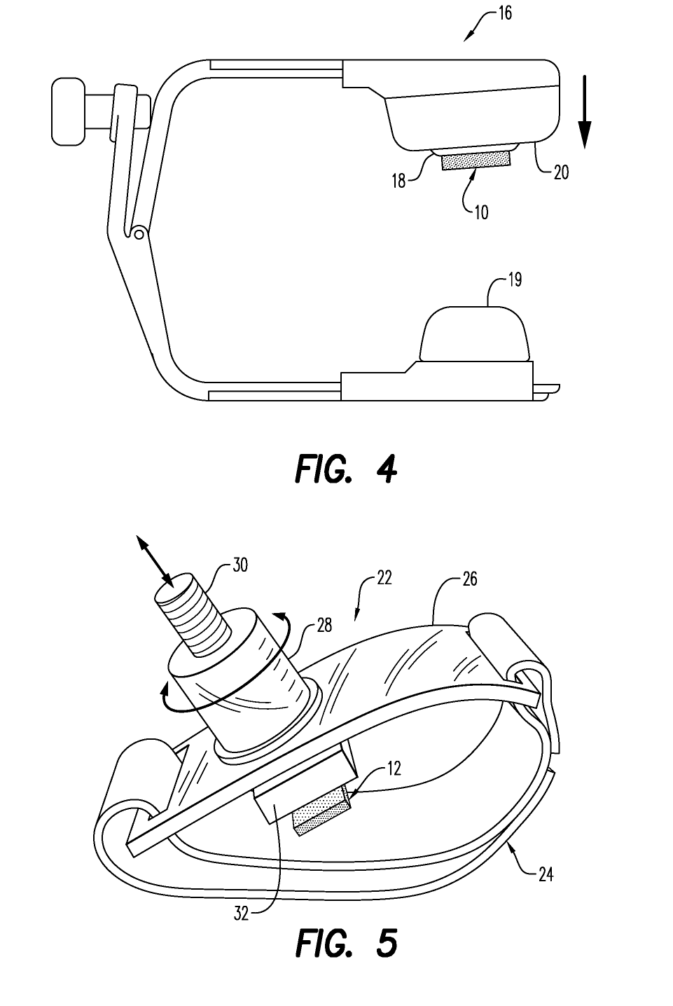

The radial arm clamp 14 provides pressure over a transdermal puncture site in an artery, such as the radial artery, to stanch the flow of blood from the artery and to induce hemostasis without otherwise overly constricting the radial artery, while permitting the clinician to perform other tasks as hemostasis is achieved. FIG. 3 shows schematically in cross section the forearm ulnar bone, radius bone, arteries, and nerves of the left forearm, as disclosed in U.S. Pat. No. 6,663,653.

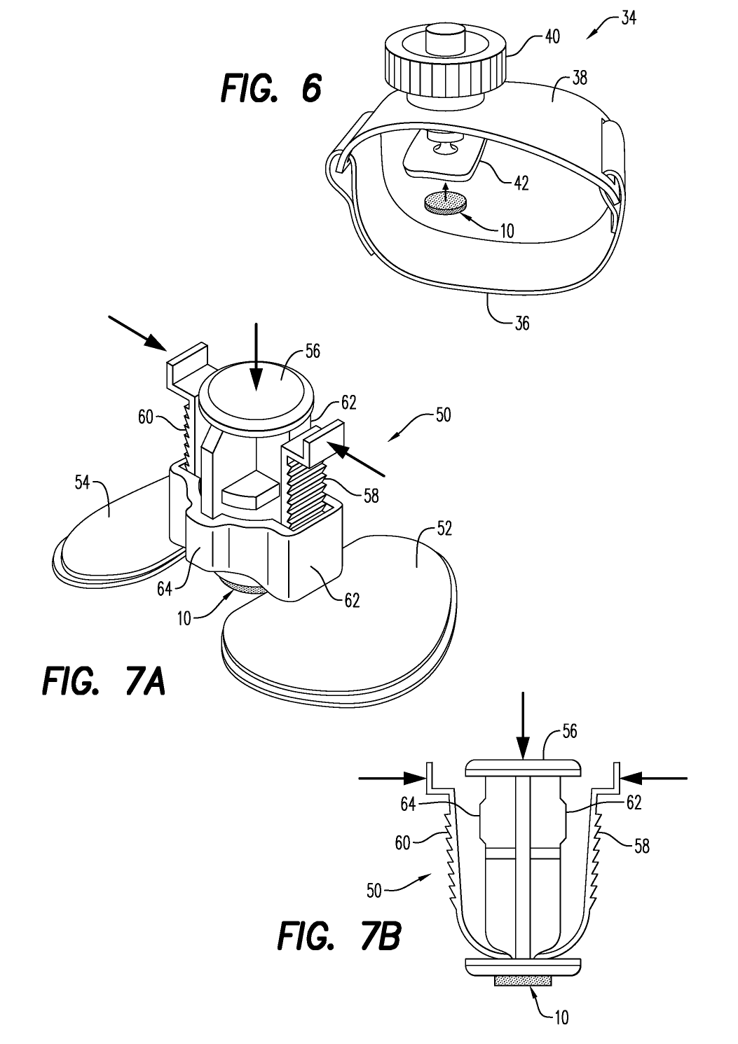

With reference to FIG. 4, an adjustable radial artery compressor 16 includes a support arm and a compression arm pivotally connected together at their proximal ends to form an adjustable C-shaped clamp. The support arm and the compression arm are pivotally connected in a hinge joint in such a way that the proximal end of the support arm extends behind the compression arm. At the distal end, i.e., the end remote from the hinge joint, the inside of the support arm is provided with a support pad 19, and an opposing compression pad 20 is provided on the distal end of the compression arm. A clamping screw is threaded through the proximal end of the support arm in such a way that the front end of the clamping screw is in contact with the compression arm. By screwing the clamping screw inwards or outwards, the distance between the support pad and the compression pad 20 can be adjusted, thereby serving as a pressure activator by controlling the compression pressure in the direction of the arrow applied on the radial artery. The tablet 10, attached to the compression pad 20 by an adhesion layer 18, contacts against the puncture in the radial artery under controlled pressure to accelerate hemostasis. (Note that, for all radial artery band embodiments, tablets 10 and 12 are substantially interchangeable.)

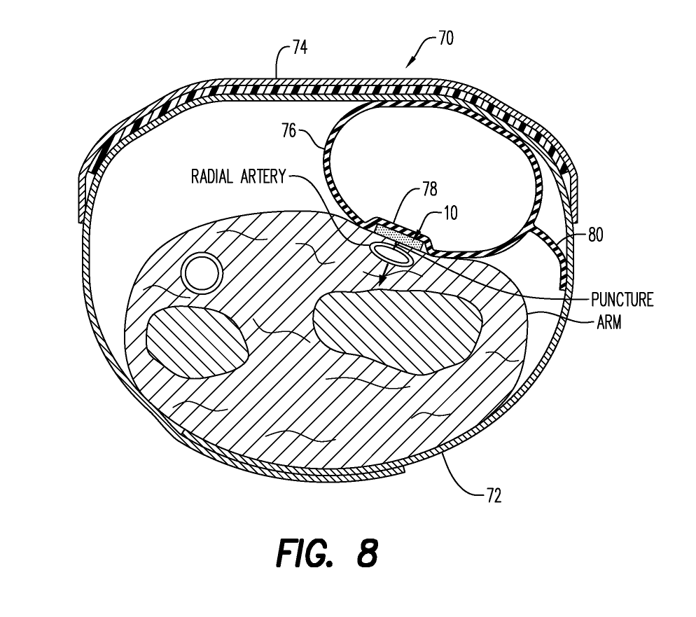

FIG. 5 illustrates the SAFERLIFE radial compression device 22 by Saferlife Products, Ltd. which stops includes a pressure actuator which bleeding by simply adjusting the band 24 or turn cap 30. The color marker 28 helps medical staff to distinguish the pressure marking bleeding point comfort. It is latex free, transparent, soft and not adhesive to the skin so that medical staff can operate precisely, effectively controlling bleeding after radial artery puncture. The hemostatic tablet 12 is attached against the compression pad 32 to make compressive contact with the puncture to reduce applied pressure against the puncture and to accelerate hemostasis.

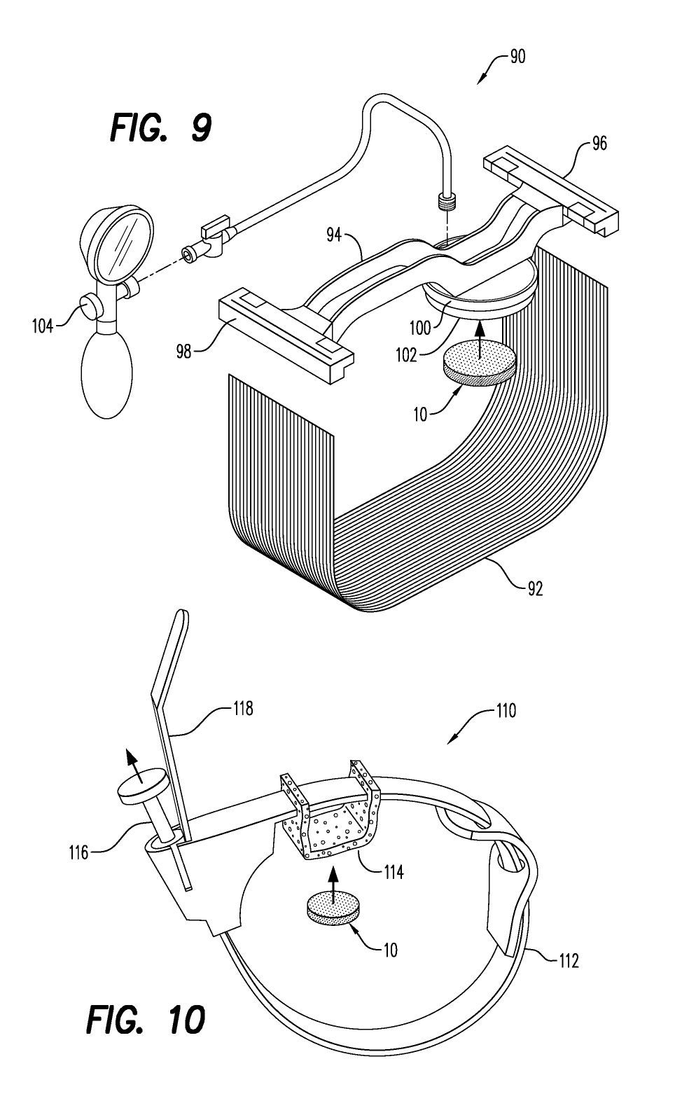

FIG. 6 illustrates another hemostasis radial artery compression device 34 by Medplus Inc. which includes a pressure actuator which assists hemostasis of the radial artery after a transradial procedure. The transparent design of support plate 38 of the device 34 enhances the precise observation of blood compression in the artery puncturing spot. Pressure can be adjusted by tightening or loosening the band 36 or screw nut 40 to enhance postoperative artery/puncture compression. A color marker assists medical staff to distinguish the pressure and makes bleeding stoppage more comfortable. The soft air cushion 42 is made of special material. There is no need to fix the patient's arm which greatly reduces a patient's pain. The device 34 ensures accuracy of pressure at the puncture spot while not overly pressing the ulnar artery making pressure to stop bleeding is more accurate. The device 34 is used to achieve hemostasis of the radial artery after an intervention or angiography operation or hemodialysis operation or invasive monitor for blood pressure. Tablet 10, attached to the air cushion, presses lightly against the puncture to greatly reduce time and pressure needed to achieve hemostasis.

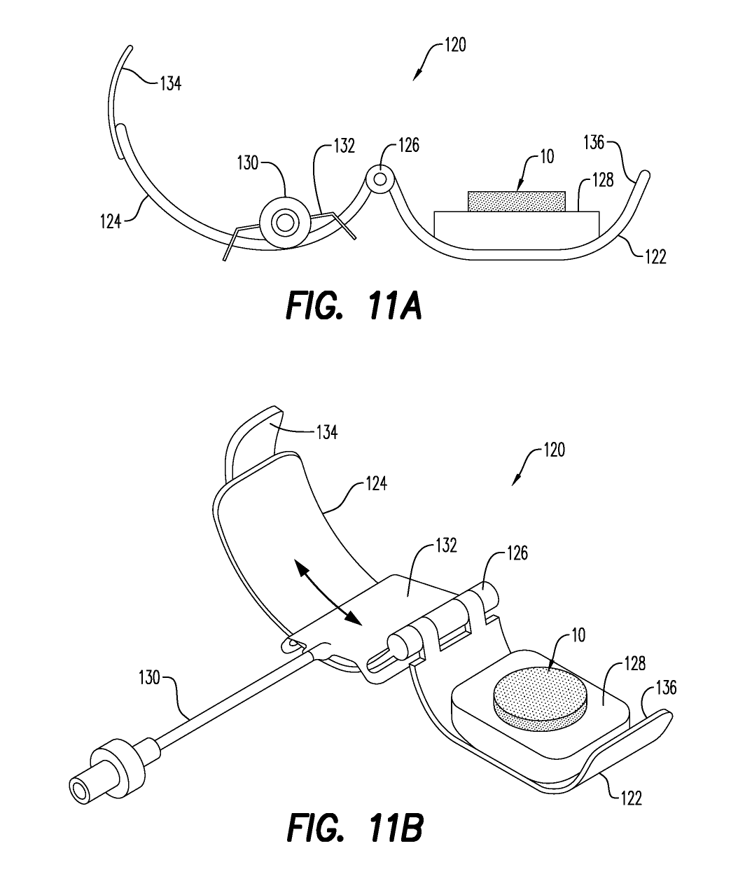

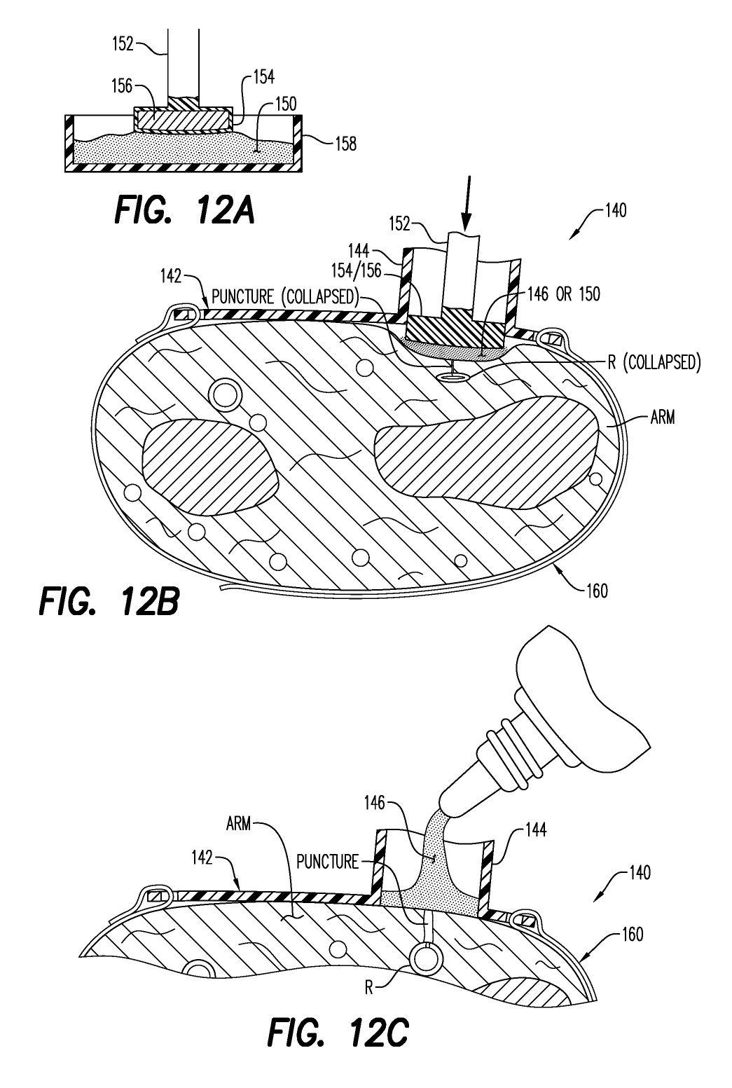

FIGS. 7A and 7B illustrate another radial artery compression device 50 disclosed in U.S. Pat. No. 8,777,982. The hemostasis device 50 comprises footplates 52 and 54, a housing 64 centrally positioned between footplates 52 and 54, and a plunger 62 acting as a pressure actuator. The engagement of the plunger 62 within the housing 64 provides for one-directional movement of the plunger 62 in the direction of the arrow by use of a ratcheting mechanism. The plunger 62 may be forced downwardly toward the wound site by applying pressure in the direction of the arrow against pad 56, but is restrained from movement upwardly in the cylinder by the combination and position of racks 58 and 60 and the corresponding pawls or racks in the cylinder. The hemostatic tablet 10 is attached to the footplate 66 to make contact against the puncture.

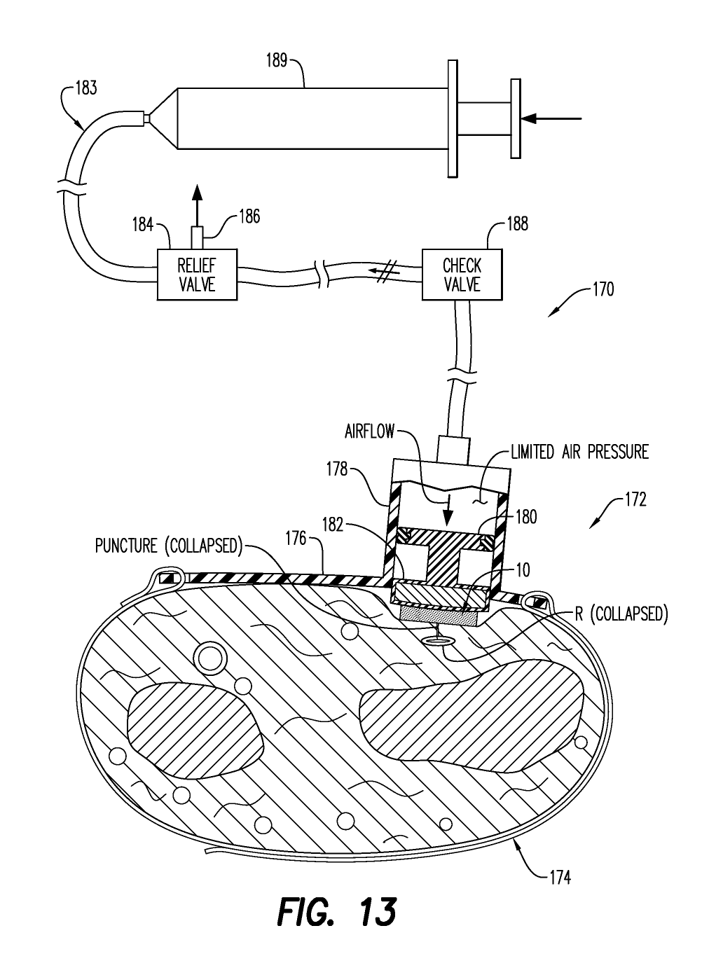

FIG. 8 is a cross section view showing a radial arm compression device 70 disclosed in U.S. Pat. No. 7,498,477 attached around an arm. The balloon 76 comes into contact with the curved outer plate 74 through the band 72. Moreover, the balloon 76 is connected to the band 72 only on one side through a connector 80, thus giving the balloon 76 a somewhat tilted orientation which enables the compression pressure F applied to the puncture site to act in an oblique direction, that is, in a direction facing the center of the wrist. The hemostatic tablet 10 is attached or held within a mating cavity 78 against the balloon 76 over the puncture and the radial artery to more rapidly achieve hemostasis.

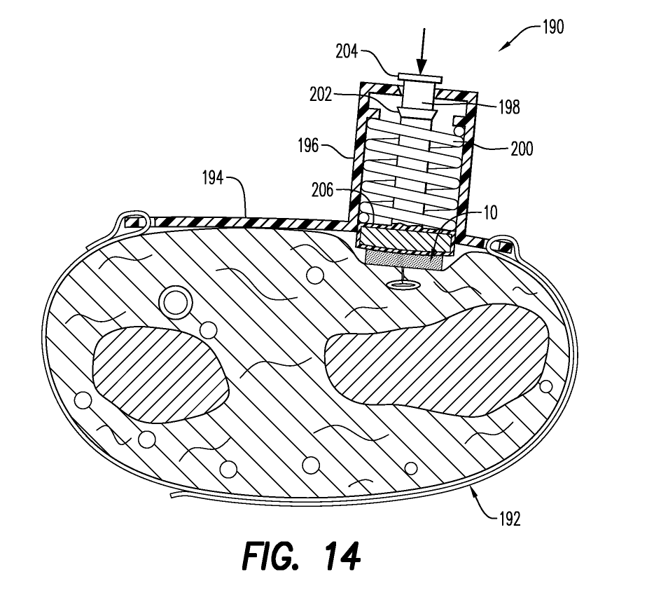

FIG. 9 shows a femoral compression device 90 disclosed in U.S. Pat. No. 6,827,727 comprising a compressor and an inflatable pressure element 102 which acts as a pressure actuator. The compressor comprises a base plate 94, a belt 92 which is adapted to be fixed around a patient's limb, and a pump 104 connected to the inflatable pressure element 102. The base plate 94 has a top portion and a bottom portion and is adapted to be fixed to the belt 92 at belt engaging ends 96 and 98. The inflatable pressure element 102 is provided at the bottom portion of the base plate 94 and is held within a support cup 100. The hemostatic tablet 10 is positioned between the inflatable pressure element or against the puncture.

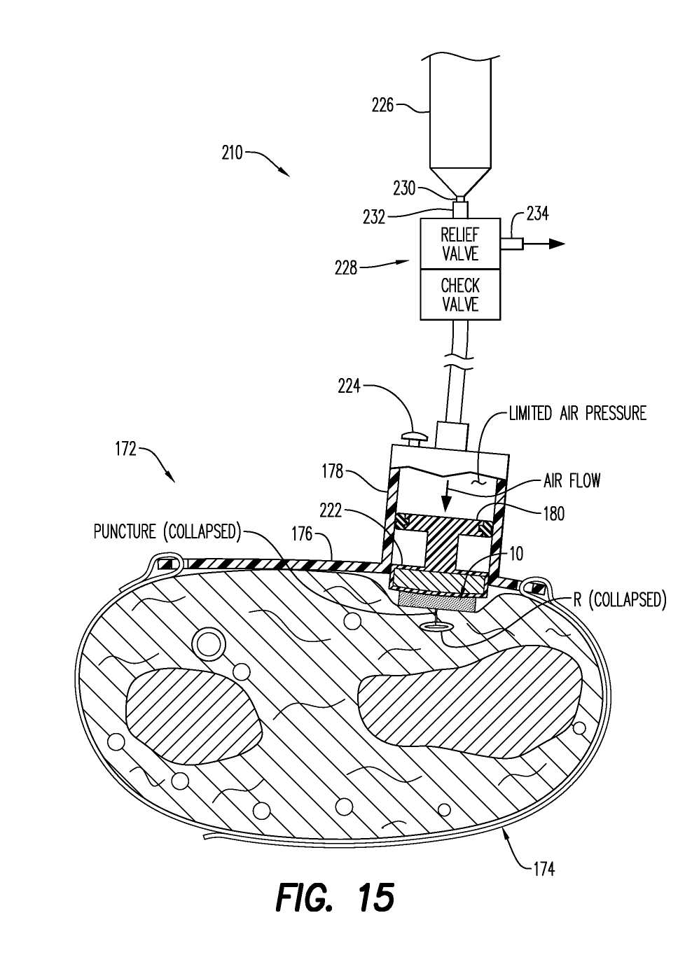

Now referring to FIG. 10, a radial artery hemostatic compression device 110 is specifically designed for economical, hands-free radial artery hemostasis for post-catheterization patients. The device 110 includes a strap 112, an adjustment knob 116 and a movable serrated band end 118 which interacts with the adjustment knob 116 to move the compression pad 114 as a pressure actuator for conveniently applying external compression to a patient's wrist. Compression pad 114 increases comfort for patients during deployment. The device 110 provides excellent access site visibility and is easily deployed by a single operator and focused compression, enabling patient ulnar flow and venous return. However, the addition of the hemostatic tablet 10 between the compression pad 114 and the puncture substantially reduces both time and pressure to achieve hemostasis.

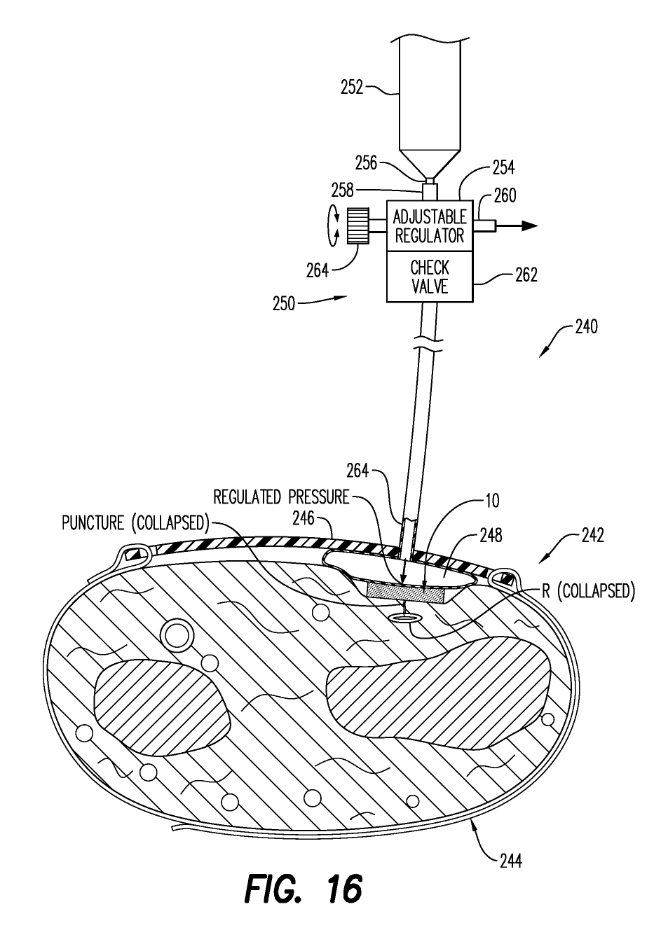

Now referring to FIGS. 11A and 11B, a sensor apparatus 130 is embodied in an integrated hemostasis band 120 disclosed in U.S. Published Application No. 2014/0012120. Two fastener components 134 and 136 comprised of interlocking clasps can open and close the apparatus 120. The body components are semi-rigid/semi-flexible bands that are substantially transparent and pivotally connected together by hinge 126. A balloon 132 is the pressure component and serves as a sensor enhancement component. Two gaps on the balloon allow the balloon to slide along the body component 124 in the direction of the arrows. An inlet provides for air to be added or released from the balloon 132. The hemostatic tablet 10 is attached to the skin-facing surface of a compressible pad 128.

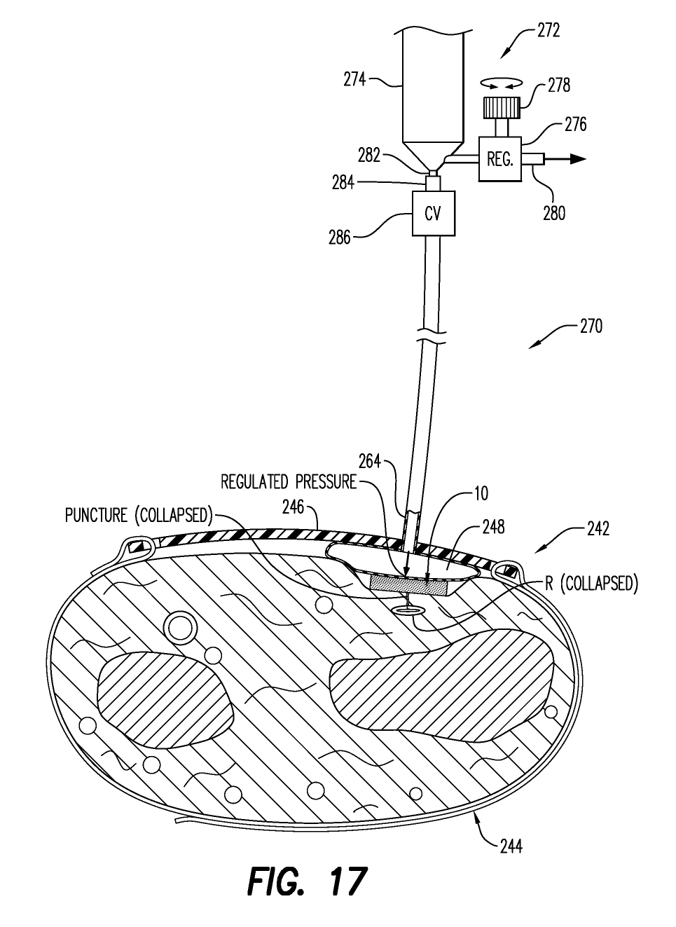

Referring now to FIGS. 12A to 12C, novel embodiments are there shown which utilize powderous WOUNDSEAL in lieu of solid STATSEAL tablets. In FIG. 12A, WOUNDSEAL plus magnetite 150 is attracted from a container 158 to the pressure pad 154 of a plunger 152 or pressure actuator having a magnet 156 held therein. The plunger 152, carrying a quantity of WOUNDSEAL plus magnetite 158 is inserted into a plunger tube 144 of a radial arm band 160 positioned over the puncture into the radial artery R as shown in FIGS. 12B and 12C. Downward pressure on the plunger 152 will collapse the puncture and radial artery which, in cooperation with the hemostatic WOUNDSEAL, will more quickly arrest blood flow. As seen in FIG. 12C, powderous WOUNDSEAL alone may alternately be poured into the plunger tube 144 over the puncture before the plunger 152 is inserted.

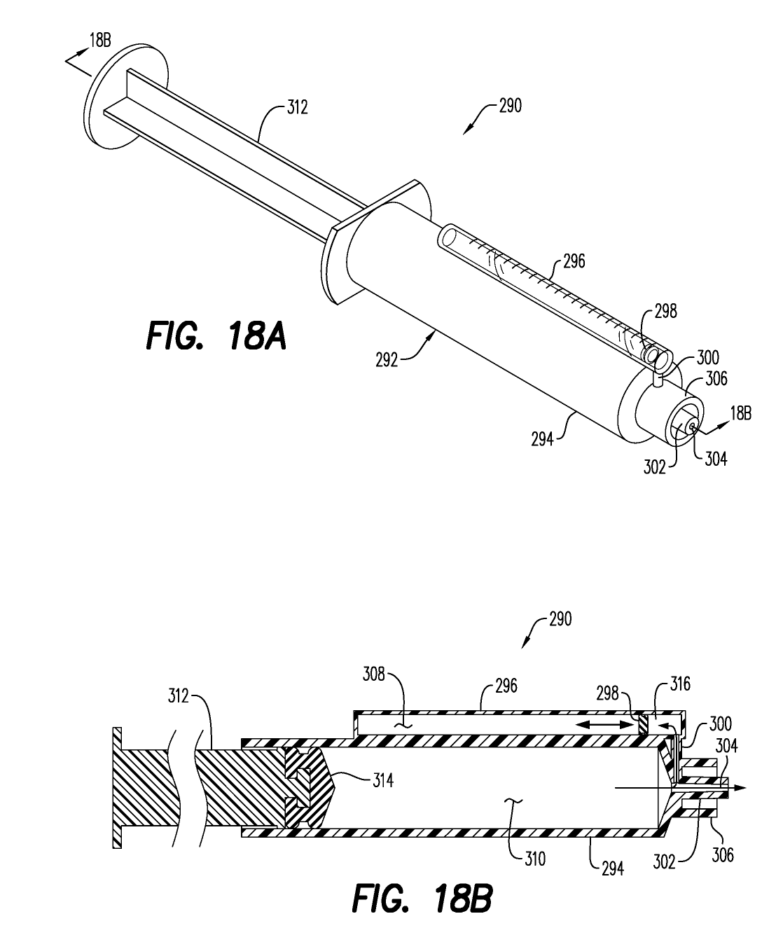

Referring to FIG. 13, to reduce excessive variation of pressure applied to close the artery and arrest blood flow from the puncture wound, it is desirable to carefully regulate that pressure by incorporating a pressure control device 183 into a radial artery hemostatic system 170. A relief valve 184 is provided to limit the pressure delivered from a syringe 189 into a plunger housing 178 of a radial band assembly 172. Thus, a controlled, predetermined pressure is delivered out of a check valve 188. Instead of putting a predetermined volume of air into the plunger housing 178, it is pressurized to a preset (or adjustable) limited air pressure. The pressure is pre-determined by the minimum force needed against the plunger 180 as the pressure actuator to achieve hemostasis at the artery in an acceptable minimum amount of time. A VELCRO strap of the radial arm band 174 of this assembly 172 holds the radial arm band 174 around the patient's wrist. The check valve 188 prevents the plunger 180 from retracting. Variation in how tightly the assembly 172 is strapped around the wrist is compensated for by controlling the travel of the plunger 180 and the force applied by the pressure pad 182 against the STATSEAL tablet 10 as automatically determined by the pressure relief valve 184.

As seen in FIG. 14, the plunger 198 of this radial artery hemostatic system 190 is driven mechanically by a calibrated compression spring 200 of a predetermined spring length and spring rate serving as the pressure actuator to apply pressure to the puncture wound. The spring 200 may be designed to compress upon application of the VELCRO strap or designed to be applied pre-compressed; a pre-activation detent 202 is released after attachment of the radial arm band assembly 192 around the arm. The spring can vary in length and spring rate for different applications. When tested with a thin flexiforce strip to measure surface pressure applied against the STATSEAL tablet 10, the pressure pad 202 provided very consistent applied pressure results over the puncture wound and STATSEAL tablet 10 even with variations in the initial tightness of the band assembly 192.

Referring to FIG. 15, a consolidated syringe activated radial artery hemostatic system 210, which is a combination of a pressure regulator 228 or pressure actuator and a radial arm band assembly 212, is provided to enhance the system 170 shown previously in FIG. 13. When the tip 230 of the syringe 226 is inserted into the pressurization port 232, fluid communication between the syringe 226 and the relief valve is established. The relief valve is then pressurized by the syringe 226 to a pre-determined pressure limited by air discharging from the pressure relief port 234. That regulated pressured air flows through a one-way check valve into the plunger housing 178 and against the plunger 180 to apply a controlled force in the direction of the arrow against the hemostatic STATSEAL tablet 10 held in place over the puncture by the radial band assembly 172 to arrest blood flow and seal the puncture.

The pressure relief-controlled balloon 248 of FIGS. 16 and 17 applies pressure against the radial artery via the STATSEAL tablet 10 with much more control and consistency than by the volume-controlled systems currently on the market. More generally, these embodiments 240 and 270 include a syringe activated check/relief valve regulator assembly 250 or 272 acting as the pressure actuator which may be utilized with any air inflated balloon type product for controlling pressure to achieve reduced time arterial hemostasis. The syringe 252 or 274 may include a pressure regulator valve 254 or 276 built into the top or barrel of the syringe for adjusting the air pressure entering the balloon 248.

In FIG. 16, one embodiment of a consolidated syringe-activated radial artery hemostatic system is shown generally at numeral 240 and is a combination pressure regulator assembly 250 and a radial arm band assembly 242. When the tip 256 of syringe 252 is inserted into the pressurization port 258, fluid communication between the syringe 226 and the adjustable pressure regulator 254 is established. When the adjustable pressure regulator 254 is pressurized by the syringe 252 to a pressure limit as established by the adjustment knob 264 of the pressure regulator 254, the pressurized air either flows into and through the check valve or, when pressure is exceeded, flows out of the pressure relief port 260. Air pressure flowing out of the check valve 262 at the predetermined pressure then flows into the balloon 248 through a balloon inlet 264 attached to, and extending from, a support plate 246 to apply the predetermined pressure against the puncture of the partially collapsed or collapsed artery R through the STATSEAL tablet 10. The check valve 262 prevents backflow of air from the balloon once the balloon has been properly pressurized. Thus, the combination of the hemostatic action of the STATSEAL tablet 10 and the regulated pressure applied by the syringe 252 into the balloon 248 against the transdermal puncture in the direction of the arrow combine to more efficiently and effectively arrest blood flow and to seal the puncture.

Referring to FIG. 17, another consolidated embodiment of a flexible VELCRO strap 244 holds the radial arm band assembly 242 around the patient's wrist. A resilient or semi-flexible support plate 246 spans over the puncture and artery R to position an integrally formed balloon inlet 264 directly over the puncture. The radial arm band assembly 242 is part of another consolidated radial artery hemostatic system 270. The system 270 also includes a pressure regulator 272 arranged in close proximity to the tip 282 of syringe 274. An adjustable pressure regulator 276 is connected to the tapering end of syringe 274 adjacent the tip 282 and provides regulated pressure adjustment through adjustment knob 278. Pressure which exceeds the preset pressure limit is discharged through the pressure relief port 280 in the direction of the arrow.

Airflow into the check valve 286 from the rigid tip 282 through the pressurization port 284 then is transferred into the collapsible balloon 248 through the balloon inlet 264. The regulated air pressure, unable to leak back into the syringe 274 by the check valve 286, then exerts a pressure in the direction of the arrow against the hemostatic tablet 10, translated against the puncture and the collapsed or partially collapsed radial artery, to effect quick and effective hemostasis of the puncture.

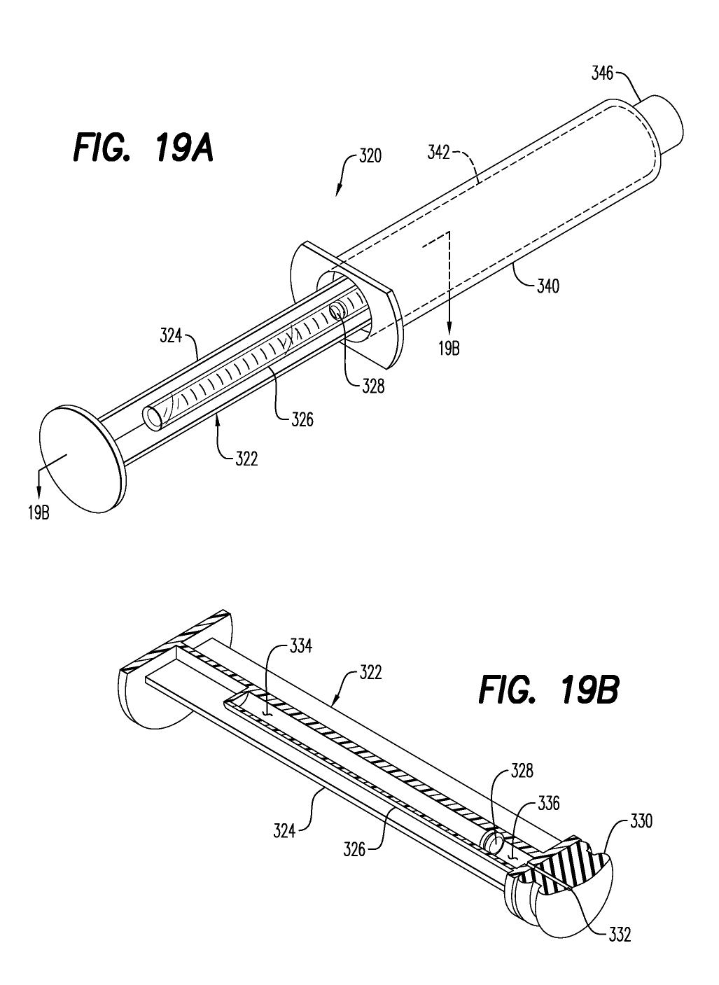

Referring to FIGS. 18A-B and 19A-B, syringe assemblies 290 and 320 are there shown for use with any air pressure-activated radial arm band adapted to receive a STATSEAL tablet 10 or 12 or WOUNDSEAL hemostatic powder. In FIGS. 18A-B, the assembly 290 includes a syringe body 292 with a syringe pressure tube 294 which defines a syringe chamber 310. A transparent sight tube 296 is attached to, and coextensive with, the outside of the syringe pressure tube 294. The syringe chamber 310 is in fluid communication with a syringe discharge pressure portion 316 of the sight tube 296 via an air transfer passage 300. A floating or movable pressure indicator 298 is sealingly and slidably positioned within the sight tube 296 whereby, when the syringe nozzle 302 and the air discharge port 304 are connected to e.g., a balloon of a radial arm band as previously described, the air pressure created in the port 304 will be the same as that produced within the syringe chamber 310 by pressing on the end of a plunger 312 sealed by a plunger seal 314 to the inner surface of the syringe chamber 310, and the syringe discharge pressure portion 316. The pressure indicator 298 will thereby be slidably moved and be viewable against pressure calibration scale printed along the sight tube 298, to thereby provide viewable indicia of the pressure created within the e.g., balloon to effect hemostasis of the bleeding puncture as previously described.

In FIGS. 19A-B, a syringe assembly 320 functions similar to the syringe assembly 290 previously described and includes a plunger assembly 322 having a plunger seal 330 for moving pressurized air through the syringe chamber 342 from the syringe nozzle 346 through an air pressure port 332 and into a e.g., a balloon. The air discharge port 332 is also in fluid communication with a syringe discharge pressure portion 336 on one side of a sealingly, slidably movable pressure indicator 328 within a transparent sight pressure tube 326 coextensive within the plunger 326. Pressure created within the syringe chamber 342 is thereby viewably reflected by the position of the movable pressure indicator 328 within the sight chamber 334.

### Protocol

The current recommended protocol for achieving hemostasis in a relatively short amount of time as compared to current practice with respect to a radial arterial vascular puncture is as follows:

Tablet Separate from Band:

1) Use a transparent dressing to secure the tablet over the radial site.

2) Apply the band.

3) Pull the sheath while inflating or tightening the band to the desired pressure
a. Ensure that the band does not occlude the ulnar artery

4) After 30 mins slowly deflate or loosen the band, and observe the site for hematoma formation.

### Tablet Attached to Band:

1) Apply the band ensuring that the tablet is centered over the radial site.

2) Pull the sheath while inflating or tightening the band to the desired pressure.
a. Ensure that the band does not occlude the ulnar artery

3) After 30 mins slowly deflate or loosen the band, and observe the site for hematoma formation.

An alternate procedure for FIGS. 16 and 17 may include:

1) Apply 200 mmHg in balloon for 15 mins

2) Reduce pressure to 100 mmHg in balloon for 15 mins

3) Reduce pressure to 50 mmHg in balloon for 15 mins

4) 0 mmHg

While a number of exemplary aspects and embodiments have been discussed above, those skilled in the art will recognize certain modifications, permutations and additions and subcombinations thereof. It is therefore intended that the following appended claims and claims hereinafter introduced are interpreted to include all such modifications, permutations, additions and subcombinations that are within their true spirit and scope.

1. In combination, a radial arm band and a hemostatic material comprising:
said hemostatic material including a hemostatic mixture of potassium ferrate and a cation ion exchange resin in powder, granular or tablet form;
said hemostatic material having improved topical hemostatic properties in the arresting of blood flow from a transdermal puncture wound into the radial artery made during transradial diagnostic or interventional catheterization procedures;
said radial arm band adapted to be wrapped around a patient's arm and positionable to hold said hemostatic material directly against the puncture wound;
said combination reducing the applied pressure needed to be applied by said radial arm band against said hemostatic material and a likelihood of harm to the radial artery, while minimizing time to achieve hemostasis of bleeding from the puncture.

2. The combination of claim 1, wherein:
said radial arm band includes an inflatable balloon attached to said radial arm band for air pressurization to apply pressure against the puncture wound and said hemostatic material.

3. The combination of claim 2, further comprising:
a syringe connectable to said balloon for controlled inflation of said balloon.

4. The combination of claim 3, further comprising:
a pressure relief valve disposed at an outlet nozzle of said syringe for limiting the air pressure from said syringe into said balloon whereby pressure applied against the hemostatic material and the puncture wound;
a check valve in fluid communication with said relief valve for maintaining air pressure in, and pressurized air from leaking from, said balloon.

5. The combination of claim 3, wherein said syringe includes:
a syringe pressure tube in fluid communication with a syringe chamber of said syringe for providing viewable indicia of an air pressure delivered into said balloon by said syringe.

6. The combination of claim 1, wherein:
said hemostatic material in powder or granular form includes magnetite;
said radial arm band includes a magnetic plunger for attracting and delivering a quantity of said hemostatic material with magnetite into a plunger tube attached to said radial arm band and positionable over the puncture wound when said radial arm band is wrapped around the arm.

7. The combination as set forth in claim 1, wherein:
said radial arm band includes a plunger which is forcibly urged by a compression spring to apply a pre-determined pressure against said hemostatic material, said plunger being positionable over the puncture wound when said radial arm band is wrapped around the arm.
